(19) United States
(12) Patent Application Publication
(10) Pub. No.: US 2016/0367745 A1
(43) Pub. Date: Dec. 22, 2016

(54) FILTER

(71) Applicants: Ying-Yeh LIU, Taoyuan City (TW); Chia-Chen CHEN, Taoyuan City (TW); Yu-Pin LlN, Taoyuan City (TW)

(72) Inventors: Ying-Yeh LIU, Taoyuan City (TW); Chia-Chen CHEN, Taoyuan City (TW); Yu-Pin LlN, Taoyuan City (TW)

(21) Appl. No.: 14/830,644

(22) Filed: Aug. 19, 2015

(30) Foreign Application Priority Data
Jun. 18, 2015 (TW) 104119718

(51) Int. Cl. A61M 1/36

(52) U.S. Cl. CPC A61M 1/3635; A61M 2205/7509; A61M 2205/7518; A61M 2205/7536

(57) ABSTRACT

A filter for a hemodialysis apparatus is provided, which includes: a first cover member, a second cover member coupled to the first cover member, and a membrane disposed in a chamber of the first cover member. The membrane has a first membrane layer and two second membrane layers respectively bonded to two opposite sides of the first membrane layer. A plurality of first ridges and second ridges are formed on a side surface of the second cover member, spaced apart from one another and having different widths so as to increase the friction force. The non-directional three-layer structure of the membrane has a high water pressure resistance, greatly reduces the chance of fluid leakage, and avoids erroneous disposition.

### BACKGROUND OF THE INVENTION

1. Field of the Invention

The present invention relates to filters, and more particularly, to a filter used for a hemodialysis apparatus.

2. Description of Related Art

A hemodialysis apparatus is generally used for patients whose kidneys don't work properly. The hemodialysis apparatus in combination with medical grade flexible tubes forms an extracorporeal blood circulation loop and enables circulation of blood outside the body of a patient. When the patient is treated with hemodialysis, the blood pressure of the patient must be monitored. Therefore, a pressure meter is provided in the extracorporeal blood circulation loop of the hemodialysis apparatus for monitoring blood pressure.

To prevent external bacteria or viruses from entering into the pressure meter through the extracorporeal blood circulation loop of the hemodialysis apparatus, a filter is provided between the pressure meter and the extracorporeal blood circulation loop. The filter has a membrane therein for filtering out bacteria or viruses and preventing blood from flowing into the pressure meter so as to protect the pressure meter against pollution. As such, a filter generally has at least two functions of: (1) blocking blood and allowing air to pass through for blood pressure detection; and (2) using the membrane to block bacteria or viruses that may be contained in the blood so as to protect the pressure meter from being contaminated by the bacteria or viruses.

To allow the membrane to be mounted inside the filter, the filter is configured to have an upper cover and a lower cover. The upper cover and the lower cover have male and female connecting portions, respectively. The upper cover and the lower cover are assembled with the membrane mounted therein and then the upper cover and the lower cover are coupled by bonding the male and female connecting portions. As such, a filter is obtained. However, during the above-described process, the membrane must be positioned in the filter in a correct direction. Otherwise, if the membrane is positioned in the filter in an erroneous direction, the membrane cannot function properly. Further, the male and female connecting portions are generally bonded through an ultrasonic welding line and a corresponding concave portion. Such a bonding method easily leads to a weak bonding (for example, non-tight bonding) and hence causes a leakage problem. Furthermore, the upper cover and the lower cover of the filter are not well designed to facilitate a user to operate the filter. For example, the upper cover and the lower cover of the filter easily slip off from hands and it is quite difficult for the user to exert forces thereon.

Therefore, how to overcome the above-described drawbacks has become critical.

### SUMMARY OF THE INVENTION

In view of the above-described drawbacks, the present invention provides a filter used for a hemodialysis apparatus, which comprises: a first cover member having a first plate body having opposite first and second surfaces, wherein a first tube body is formed on the first surface of the first plate body and a first protruding portion is formed on the second surface of the first plate body to define a chamber; a second cover member having a second plate body having opposite third and fourth surfaces and a side surface connecting the third and fourth surfaces, wherein a second tube body is formed on the third surface of the second plate body, a recessed portion is formed on the fourth surface of the second plate body and corresponds in shape to the first protruding portion, and a plurality of first ridges and second ridges are formed on the side surface of the second plate body, spaced apart from one another and having different widths, the second cover member being coupled to the first cover member with the first protruding portion of the first cover member being received in the recessed portion of the second cover member; and a membrane disposed in the chamber of the first cover member, wherein the membrane has a first membrane layer and two second membrane layers respectively bonded to two opposite sides of the first membrane layer.

The first ridges and the second ridges that are formed on the side surface of the second plate body facilitate a user to hold the side surface of the second plate body for operation, and the second plate body is not easy to slip off from hands of the user. Further, at least a protruding point can be formed on one of the first protruding portion and the recessed portion and at least a recessed point corresponding to the protruding point can be formed on the other of the first protruding portion and the recessed portion so as to increase the bonding area and hence strengthen the bonding between the first protruding portion and the recessed portion after an ultrasonic welding process. As such, the present invention prevents the problem of leakage that otherwise may be caused by a non-tight bonding. Furthermore, since the membrane is non-directional, the present invention prevents the membrane from being disposed in the filter in an erroneous direction as in the prior art. In addition, the multi-layer structure of the membrane greatly reduces the chance of fluid leakage, facilitates the fabrication process and improves the assembly yield and efficiency.

### BRIEF DESCRIPTION OF DRAWINGS

FIG. 1 is a schematic perspective view of a filter of the present invention;

FIG. 2 is a schematic exploded view of the filter of the present invention;

FIG. 3 is a schematic cross-sectional view of the filter of the present invention;

FIGS. 4A and 4B are schematic views showing the arrangement of first flanges in the chamber of the filter according to different embodiments of the present invention;

FIGS. 5A and 5B are schematic views showing the shape of a second plate body of the filter according to different embodiments of the present invention; and

FIG. 6 is a schematic cross-sectional view of the filter according to another embodiment of the present invention.

### DETAILED DESCRIPTION OF PREFERRED EMBODIMENTS

The following illustrative embodiments are provided to illustrate the disclosure of the present invention, these and other advantages and effects can be apparent to those in the art after reading this specification.

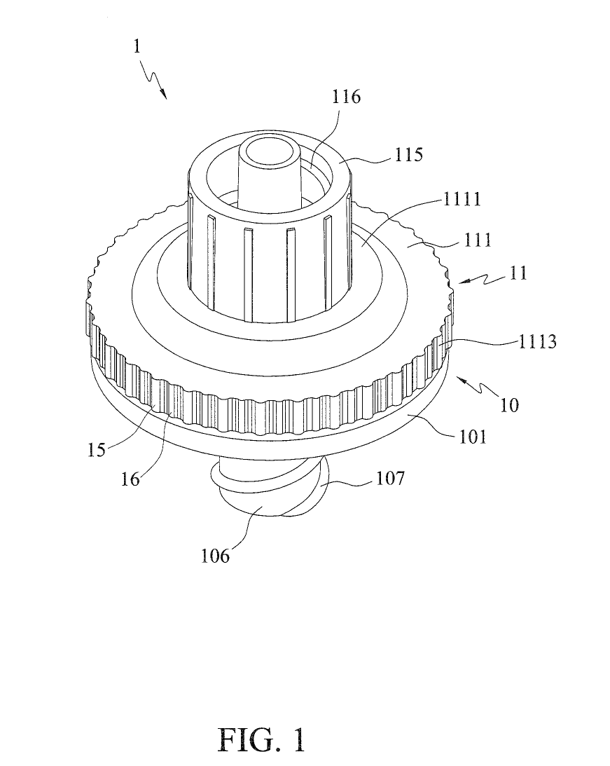

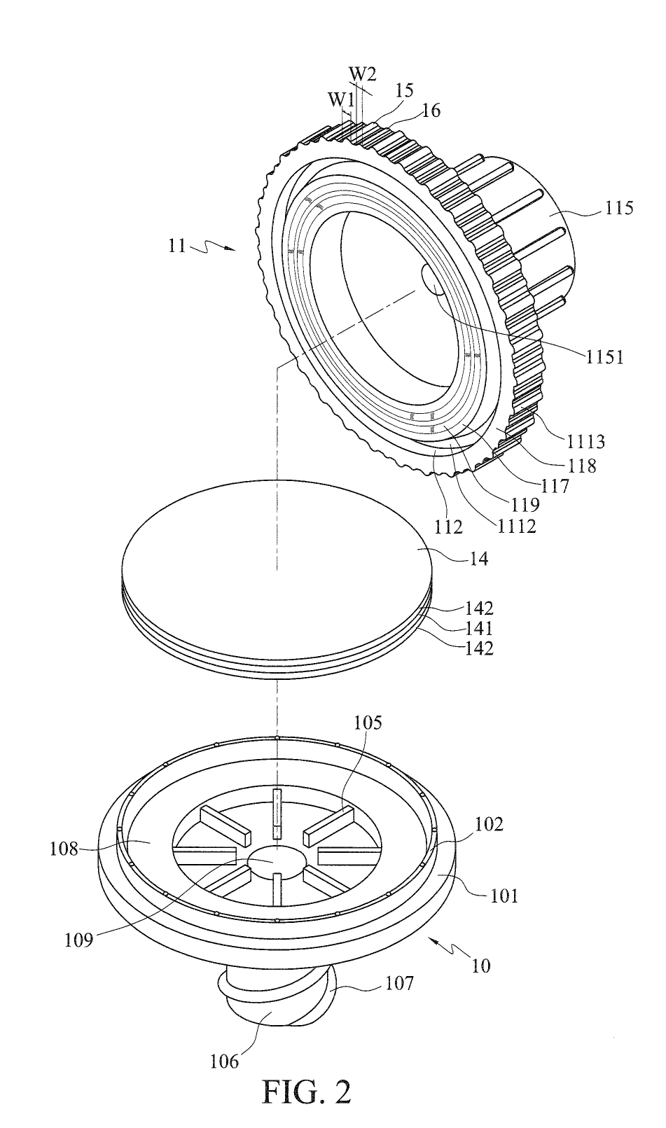

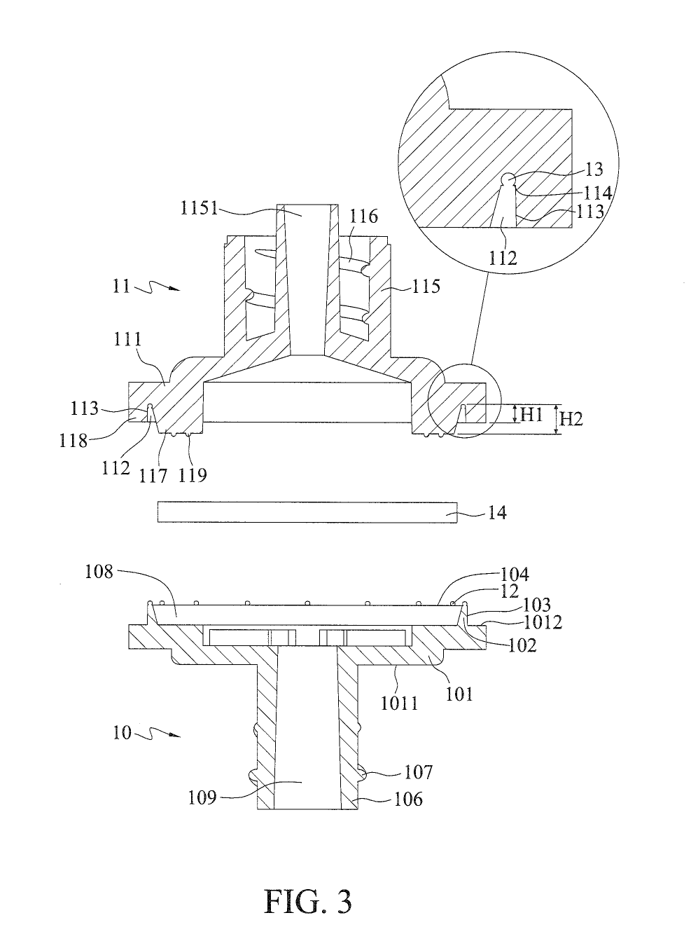

FIG. 1 is a schematic perspective view of a filter 1 of the present invention, FIG. 2 is a schematic exploded view of the filter 1, and FIG. 3 is a schematic cross-sectional view of the filter 1. Referring to FIGS. 1 to 3, the filter 1 is used for a hemodialysis apparatus. The hemodialysis apparatus in combination with medical grade flexible tubes forms an extracorporeal blood circulation loop, and a pressure meter is mounted in the extracorporeal blood circulation loop for monitoring blood pressure. The filter 1 of the present invention is mounted to the flexible tube between the pressure meter and the extracorporeal blood circulation loop.

The filter 1 has a first cover member 10, a second cover member 11, and a membrane 14. The first cover member 10 and the second cover member 11 can be made of a thermoplastic material such as PVC, PC, PE or PP. In particular, the first cover member 10 and the second cover member 11 can be made of a medical grade thermoplastic material.

The first cover member 10 has a first plate body 101 and a first tube body 106. The first plate body 101 has a first surface 1011 and a second surface 1012 opposite to the first surface 1011. The first tube body 106 extends outward from the center of the first surface 1011 of the first plate body 101, and a hollow channel 109 is formed inside the first tube body 106. A first protruding portion 102 is formed on the second surface 1012 of the first plate body 101, and a chamber 108 is defined by the first protruding portion 102 together with the second surface 1012 of the first plate body 101.

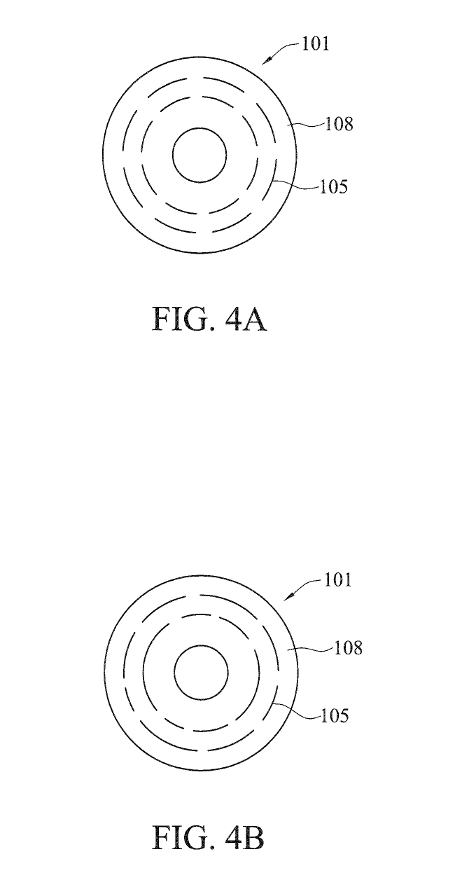

In an embodiment, a plurality of first flanges 105 are formed in the chamber 108 and arranged in a shape like a Chinese character “ [symbol] ”, as shown in FIG. 2. In other embodiments, the first flanges 105 are arranged in a shape of symmetric concentric circles, as shown in FIG. 4A, or arranged in a shape of asymmetric concentric circles, as shown in FIG. 4B. “Symmetric” means the gaps between adjacent first flanges 105 along the circumferential directions of the concentric circles are aligned along the radial direction of the concentric circles. The concentric circles can provide a preferred supporting function to the membrane. In addition, the first flanges 105 can be arranged in a cross shape.

The second cover member 11 has a second plate body 111 and a second tube body 115. The second plate body 111 has a third surface 1111, a fourth surface 1112 opposite to the third surface 1111 and a side surface 1113 connecting the third surface 1111 and the fourth surface 1112. The second tube body 115 extends outward from the center of the third surface 1111 of the second plate body 111, and a hollow channel 1151 is formed in the second tube body 115. Further, a recessed portion 112 is formed on the fourth surface 1112 of the second plate body 111 and corresponds in shape to the first protruding portion 102. But the height of the first protruding portion 102 is greater than the depth of the recessed portion 112. For example, the height of the first protruding portion 102 is 1.63 mm and the depth of the recessed portion 112 is 0.95 mm. By bonding the recessed portion 112 of the second cover member 11 with the first protruding portion 102 of the first cover member 10, the second cover member 11 is coupled to the first cover member 10 so as to form the filter 1. After the second cover member 11 is coupled to the first cover member 10, the hollow channel 109 of the first tube body 106 and the hollow channel 1151 of the second tube body 115 are coaxial and communicate with one another, and the first protruding portion 102 of the first cover member 10 is received in the recessed portion 112 of the second cover member 11.

In an embodiment, the first protruding portion 102 of the first plate body 101 and the recessed portion 112 of the second plate body 111 have a ring shape. In other embodiments, the first protruding portion 102 of the first plate body 101 and the recessed portion 112 of the second plate body 111 have a rectangular shape, a triangular shape and so on.

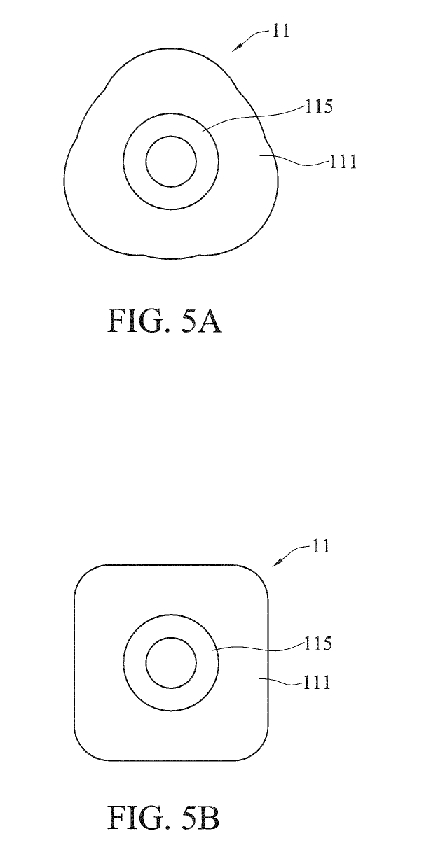

In an embodiment, the second plate body 111 has a circular shape. Alternatively, the second plate body 111 has a flower shape, as shown in FIG. 5A, or a rectangular shape, as shown in FIG. 5B.

In an embodiment, the first tube body 106 is a female luer type connector and the second tube body 115 is a male luer type connector. The female luer type connector and the male luer type connector can be luer-lock or luer-slip connectors. For example, referring to FIGS. 1 to 3, the first tube body 106 has external threads 107 formed on an outer surface thereof and the second tube body 115 has internal threads 116 formed on an inner surface thereof. That is, the connectors are luer-lock connectors. The external threads 107 and the internal threads 116 allow the first tube body 106 and the second tube body 115 to be securely connected between the pressure meter and the flexible tube of the hemodialysis apparatus without unintended detachment.

A plurality of first ridges 15 and a plurality of second ridges 16 are formed on the side surface 1113 of the second plate body 111, spaced apart from one another and having different widths. In an embodiment, the first ridges 15 have a width W1 and the second ridges 16 have a width W2. The ratio of the width W1 of the first ridges 15 to the width W2 of the second ridges 16 is in a range between 2:1 and 5:1. Further, the height of the first ridges 15 protruding from the side surface 1113 can be greater than or equal to the height of the second ridges 16 protruding from the side surface 1113.

The membrane 14 is disposed in the chamber 108 defined by the first protruding portion 102 of the first cover member 10. The outer diameter of the membrane 14 is substantially equal to the inner diameter of the chamber 108. As such, the membrane 14 is fixed by the chamber 108.

In an embodiment, the membrane 14 is a composite membrane having a three-layer structure. In particular, the membrane 14 has a first membrane layer 141, and two second membrane layers 142 bonded to two opposite sides of the first membrane layer 141, respectively. The first membrane layer 141 is made of non-woven fabric, and the second membrane layers 142 are made of ePTFE, PVDF or UHMWPE. The second membrane layers 142 are porous. The pore size of the second membrane layers 142 is below 0.26 μm, preferably between 0.01 and 0.2 μm.

The first protruding portion 102 of the first cover member 10 and the recessed portion 112 of the second cover member 11 are bonded through an ultrasonic welding process. In the ultrasonic welding process, a high frequency signal causes molecular friction on the interface between the first protruding portion 102 and the recessed portion 112 and hence produces local high temperature to melt the thermoplastic material of the interface between the first protruding portion 102 and the recessed portion 112. When the melted thermoplastic material cools and solidifies, the first protruding portion 102 and the recessed portion 112 are bonded together. As such, after the first cover member 10 and the second cover member 11 are coupled through ultrasonic welding to form the filter 1, the membrane 14 positioned between the first cover member 10 and the second cover member 11 can effectively block blood in the flexible tube of the extracorporeal blood circulation loop from entering into the pressure meter so as to protect the pressure meter against pollution. On the other hand, the membrane 14 allows air to pass through so as for the pressure meter to detect pressure variation.

In an embodiment, a second protruding portion 117 and a third protruding portion 118 are formed on the fourth surface 1112 of the second plate body 111 to define the recessed portion 112. In the case both the first protruding portion 102 and the corresponding recessed portion 112 have a ring shape, the second protruding portion 117 and the third protruding portion 118 also have a ring shape. In the present embodiment, the height H2 of the second protruding portion 117 is greater than the height H1 of the third protruding portion 118. As such, after the second cover member 11 is coupled to the first cover member 10, the second protruding portion 117 tightly presses an edge of the membrane 14 in the chamber 108 of the first cover member 10, thereby enhancing the fixing effect of the membrane 14. In other embodiments, the height H2 of the second protruding portion 117 is equal to the height H1 of the third protruding portion 118. Even in this case, since the membrane has a certain height, the second protruding portion 117 can tightly press an edge of the membrane 14 in the chamber 108 of the first cover member 10.

In another embodiment, at least a second flange 119 is formed on a top surface of the second protruding portion 117. The second flange 119 has a ring shape and is concentric with the second protruding portion 117. After the second cover member 11 is coupled to the first cover member 10, the second flange 119 can tightly press an edge of the membrane 14 in the chamber 108 of the first cover member 10. Further, the second flange 119 effectively reduces the chance of leakage of blood in the filter 1.

Referring to FIG. 3, to strengthen the bonding between the first cover member 10 and the second cover member 11, at least a protruding point 12 or recessed point 13 is formed on the first protruding portion 102 or the recessed portion 112. If the protruding point 12 is formed on the first protruding portion 102, the recessed point 13 is formed on the recessed portion 112 and corresponds to the protruding point 12. Otherwise, if the recessed point 13 is formed on the first protruding portion 102, the protruding point 12 is formed on the recessed portion 112 and corresponds to the recessed point 13.

In an embodiment, the protruding point 12 is formed on a top surface 104 of the first protruding portion 102, and the recessed point 13 is formed on a top surface 114 of the recessed portion 112. Alternatively, the protruding point 12 can be formed on a side 103 of the first protruding portion 102, and the recessed point 13 can be formed on a side 113 of the recessed portion 112. The protruding point 12 and the corresponding recessed point 13 can have a circular shape or a triangular shape. It should be noted that the number, position and shape of the protruding point 12 and the recessed point 13 can be varied according to the practical need.

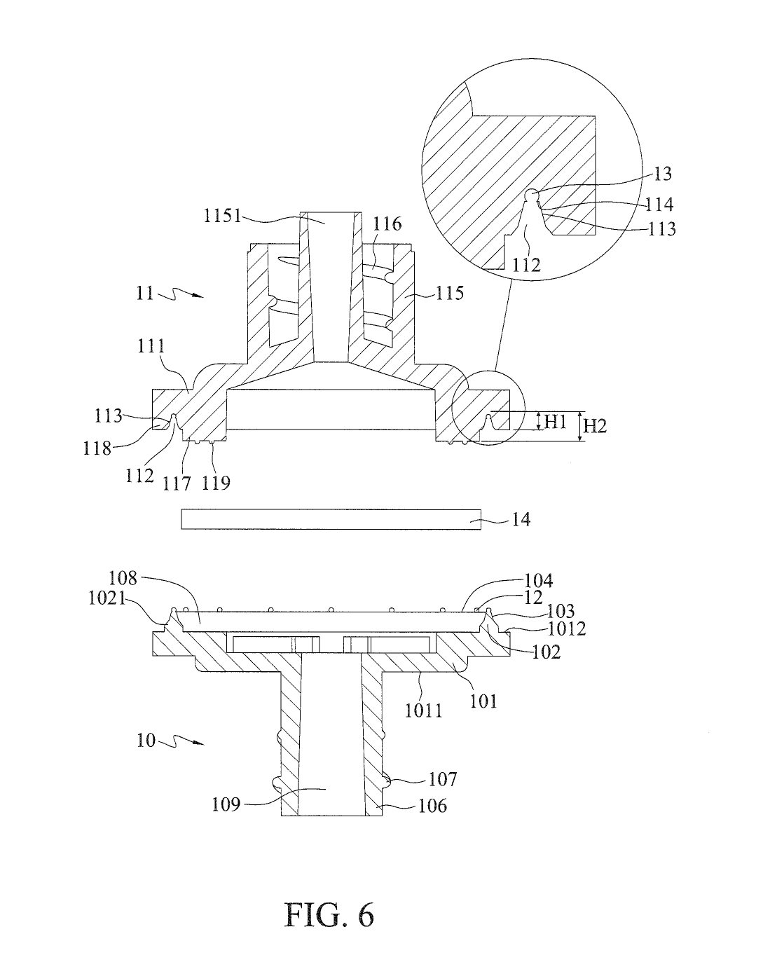

FIG. 6 shows the filter according to another embodiment of the present invention. Referring to FIG. 6, a carrying portion 1021 is formed on and protrudes from the side 103 of the first protruding portion 102 and located on the second surface 1012 of the first plate body 101. The contact portion between the carrying portion 1021 and the recessed portion 112 of the second cover member 11 is an inclined surface. After the second cover member 11 is coupled to the first cover member 10, the carrying portion 1021 protruding from the side 103 is not completely received in the recessed portion 112 of the second cover member 11. Therefore, the carrying portion 1021 is sandwiched between the first plate body 101 and the second plate body 111 and a gap is formed between the first cover member 10 and the second cover member 11, thus increasing the assembly accuracy and yield and causing the membrane 14 to be tightly pressed without being damaged by an insufficient or excessive bonding pressure between the first cover member 10 and the second cover member 11. Also, the bonding between the first cover member 10 and the second cover member 11 is strengthened to reduce liquid or air leakage.

According to the present invention, at least a protruding point 12 is formed on one of the first protruding portion 102 and the recessed portion 112 and at least a recessed point 13 corresponding to the protruding point 12 is formed on the other of the first protruding portion 102 and the recessed portion 112. The protruding point 12 and the recessed point 13 facilitate to increase the contact area between the first cover member 10 and the second cover member 11. During the ultrasonic welding process, the increased contact area causes a larger area of thermoplastic material to be melted and a larger area of melted thermoplastic material to cool and solidify, thereby strengthening the bonding between the first cover member 10 and the second cover member 11. As such, the present invention prevents the problem of leakage that otherwise may be caused by a non-tight bonding between the first cover member 10 and the second cover member 11.

Further, a plurality of first ridges 15 and second ridges 16 are formed on the side surface 1113 of the second plate body 111, spaced apart from one another and having different widths. The first ridges 15 and the second ridges 16 increase the coefficient of friction of the side surface 1113 of the second plate body 111. Therefore, the side surface 1113 of the second plate body 111 can be easily held by a user for operation and the second plate body 111 is not easy to slip off from hands of the user. For example, when an ASTM D1894 testing method is used, the conventional filter has a static coefficient of friction of 0.357±0.027 while the filter of the present invention has a static coefficient of friction of 0.416±0.012, which is much higher than the conventional filter. The ratio of the width W1 of the first ridges 15 to the width W2 of the second ridges 16 is in a range between 2:1 and 5:1. Preferably, the ratio of the width W1 of the first ridges 15 to the width W2 of the second ridges 16 is 2:1 taking into account of visual effect and operability.

Furthermore, the membrane 14 is a composite membrane having a three-layer structure. The first membrane layer 141 in the middle is made of non-woven fabric and the two second membrane layers 142 bonded to the two opposite sides of the first membrane layer 141 are made of ePTFE, PVDF or UHMWPE. The three-layer structure of the membrane 14 has a high water pressure resistance (about 2 Kg/cm²) and greatly reduces the chance of fluid leakage. Further, since the membrane 14 is non-directional, the present invention prevents the membrane 14 from being disposed in the filter 1 in an erroneous direction as in the prior art. Compared with the conventional filter that has a water pressure resistance of 0.5 Kg/cm² and is directional, the filter of the present invention avoids erroneous disposition of the membrane, greatly reduces the chance of fluid leakage, facilitates the fabrication process and increases the assembly yield and efficiency. The above-described water pressure resistance values are measured according to JIS L 1092 standard.

The above-described descriptions of the detailed embodiments are only to illustrate the preferred implementation according to the present invention, and it is not to limit the scope of the present invention. Accordingly, all modifications and variations completed by those with ordinary skill in the art should fall within the scope of present invention defined by the appended claims.

What is claimed is:

1. A filter used for a hemodialysis apparatus, comprising:
a first cover member having a first plate body having opposite first and second surfaces, wherein a first tube body is formed on the first surface of the first plate body and a first protruding portion is formed on the second surface of the first plate body to define a chamber;
a second cover member having a second plate body having opposite third and fourth surfaces and a side surface connecting the third and fourth surfaces, wherein a second tube body is formed on the third surface of the second plate body, a recessed portion is formed on the fourth surface of the second plate body and corresponds in shape to the first protruding portion, and a plurality of first ridges and second ridges are formed on the side surface of the second plate body, spaced apart from one another and having different widths, the second cover member being coupled to the first cover member with the first protruding portion of the first cover member being received in the recessed portion of the second cover member; and
a membrane disposed in the chamber of the first cover member, wherein the membrane has a first membrane layer and two second membrane layers respectively bonded to two opposite sides of the first membrane layer.

2. The filter of claim 1, wherein a ratio of the width of the first ridges to the width of the second ridges is in a range of 2:1 to 5:1.

3. The filter of claim 1, wherein the first membrane layer is made of non-woven fabric, and the second membrane layers are made of ePTFE, PVDF or UHMWPE and have a pore size between 0.01 and 0.2 μm.

4. The filter of claim 1, wherein the first tube body is a female luer type connector and the second tube body is a male luer type connector, the first tube body and the second tube body being coaxial after the second cover member is coupled to the first cover member, and wherein the female luer type connector and the male luer type connector are luer-lock or luer-slip connectors.

5. The filter of claim 1, wherein a plurality of first flanges are formed in the chamber in a cross shape, a shape like a Chinese character “ [symbol] ” or a shape of symmetric concentric circles or asymmetric concentric circles.

6. The filter of claim 1, wherein a second protruding portion and a third protruding portion are further formed on the fourth surface of the second plate body to define the recessed portion, and the second plate body has a circular shape, a rectangular shape or a flower shape.

7. The filter of claim 6, wherein the height of the second protruding portion is greater than or equal to the height of the third protruding portion, and the second protruding portion, the third protruding portion and the recessed portion have a ring shape.

8. The filter of claim 6, wherein at least a second flange is formed on a top surface of the second protruding portion and concentric with the second protruding portion so as to tightly press an edge of the membrane in the chamber of the first cover member after the second cover member is coupled to the first cover member.

9. The filter of claim 1, wherein at least a protruding point is formed on one of the first protruding portion and the recessed portion, and at least a recessed point corresponding to the protruding point is formed on the other of the first protruding portion and the recessed portion, the protruding point or the recessed point is positioned on a side or a top surface of the first protruding portion or the recessed portion, and the protruding point and the recessed point have a circular shape or a triangular shape.

10. The filter of claim 1, wherein a carrying portion is formed on and protrudes from a side of the first protruding portion in a manner that after the second cover member is coupled to the first cover member, the carrying portion is not received in the recessed portion of the second cover member and a gap is formed between the first cover member and the second cover member.
